(19) United States
(12) Patent Application Publication
(10) Pub. No.: US 2005/0158272 A1
(43) Pub. Date: Jul. 21, 2005

(54) Methods, compositions and devices for embolizing body lumens

(75) Inventors: Robert G. Whirley, Santa Rosa, CA (US); Gerald R. Martin, Windsor, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TriVascular, Inc., Santa Rosa, CA (US)

(21) Appl. No.: 11/031,311

(22) Filed: Jan. 7, 2005

Related U.S. Application Data
(60) Provisional application No. 60/534,638, filed on Jan. 7, 2004.

(51) Int. Cl. A61K 31/74

(52) U.S. Cl. 424/78.18

(57) ABSTRACT

The present invention provides embolic compositions, methods, and devices for embolizing a body lumen. In one embodiment, the embolic composition comprises a mixture of polyethylene glycol diacrylate (PEGDA), pentaerythritol tetra(3-mercaptopropionate), and a physiologically acceptable buffer solution.

### CROSS REFERENCE TO RELATED APPLICATIONS

The present application claims benefit to U.S. Provisional Patent Application Ser. No. 60/534,638, entitled “Methods, Compositions, and Devices for Embolizing Body Lumens,” filed on Jan. 7, 2004, the complete disclosure of which is incorporated herein by reference.

### BACKGROUND OF THE INVENTION

The present invention relates generally to medical devices and methods. More specifically, the present invention relates to the embolization of target sites of body lumens, such as vascular and non-vascular body lumens.

Embolization of a body lumen such as a blood vessel or organ may be used to treat a variety of maladies, including, by way of example only, controlling bleeding caused by trauma, preventing profuse blood loss during an operation requiring dissection of blood vessels, obliterating a portion of a whole organ having a tumor, blocking the blood flow into abnormal blood vessel structures such as arterio-venous malformations (AVM), arteriovenous fistulae (AVF) and aneurysms, and blocking the passage of fluids or other materials through various body lumens. For such treatments, a variety of embolization technologies have been proposed, including for example mechanical means (including particulate technology), and liquid and semi-liquid technologies. The particular characteristics of such technologies (such as, e.g., the size of particles, radiopacity, viscosity, mechanism of occlusion, biological behavior and possible recanalization versus permanent occlusion, the means by which the material is delivered to the target body site, etc.), are factors used by the physician in determining the most suitable therapy for the indication to be treated.

Of the mechanical and particulate embolization technologies, the most prevalent include detachable balloons, macro- and microcoils, gelfoam and polyvinyl alcohol sponges (such as IVALON, manufactured and sold by Ivalon, Inc. of San Diego, Calif.), and microspheres. For example, one embolization technique uses platinum and stainless steel microcoils. However, significant expertise is required to choose a proper coil size for the malady prior to delivery. Moreover, many anatomical sites are not suitable for microcoils, and removal of microcoils has proved in certain circumstances difficult.

Liquid and semi-liquid embolic compositions include viscous occlusion gels, collagen suspensions, and cyanoacrylate (n-butyl and iso-butyl cyanoacrylates). Of these, cyanoacrylates have an advantage over other embolic compositions in their relative ease of delivery and in the fact that they are some of the only liquid embolic compositions currently available to physicians. However, the constituent cyanoacrylate polymers have the disadvantage of being biodegradable. Moreover, the degradation product, formaldehyde, is highly toxic to the neighboring tissues. See Vinters et al. “The histotoxicity of cyanoacrylate: A selective review”, Neuroradiology, 1985; 27:279-291. Another disadvantage of cyanoacrylate materials is that the polymer will adhere to body tissues and to the tip of the catheter. Thus, physicians must retract the catheter immediately after injection of the cyanoacrylate embolic composition or risk adhesion of the cyanoacrylate and the catheter to tissue such as blood vessels.

Another class of liquid embolic compositions is precipitative materials, which was invented in the late 1980's. See Sugawara et al., “Experimental investigations concerning a new liquid embolization method: Combined administration of ethanol-estrogen and polyvinyl acetate”, Neuro. Med. Chir. (Tokyo) 1993; 33:71-76; Taki et al., “A new liquid material for embolization of arterio-venous malformations”, AJNR 1990; 11:163-168; Mandai et al., “Direct thrombosis of aneurysms with cellulous acetate polymer: Part I: Results of thrombosis in experimental aneurysms”, J. Neurosurgery 1992; 77:497-500. These materials employ a different mechanism in forming synthetic emboli than do the cyanoacrylate materials. Cyanoacrylate glues are monomeric and rapidly polymerize upon contact with blood. On the other hand, precipitative materials are pre-polymerized chains that precipitate into an aggregate upon contact with blood.

In the precipitation method, the polymer is dissolved in a solvent that is miscible with blood. Upon contact with the blood, the solvent is diluted and the water-insoluble polymer precipitates. Ideally, the precipitate forms a solid mass and thus occludes the vessel. The first such precipitative material used in this way was polyvinyl acetate (PVAc). Also, poly(ethylene-co-vinyl alcohol) (EVAL) and cellulose acetate (CA) dissolved in 100% dimethyl sulfoxide (DMSO) have also been used in clinical procedures. See Taki et al., “A new liquid material for embolization of arteriovenous malformations”, AJNR 1990; 11: 163-168 and Mandai et al., “Direct thrombosis of aneurysms with cellulose polymer: Part I: Results of thrombosis in experimental aneurysm”, J. Neurosurgery 1992; 77:497-500. Partially hydrolyzed polyvinyl acetate in, e.g., ethanol, is also an available member of this class.

While the conventional embolization therapies have had some success, improvements are still needed.

### BRIEF SUMMARY OF THE INVENTION

The present invention relates generally to methods, compositions, and devices for embolizing a body lumen. The present invention comprises depositing a multi-component liquid embolic composition into the body lumen and allowing the embolic composition to cure so as to embolize the target site in the body lumen.

The embolic compositions of the present invention typically cross-link and polymerize in vivo at the target site in the body lumen. Unlike conventional embolic compositions, the embolic compositions of the present invention will polymerize independent of the environment of the target site and do not require an external triggering mechanism to start the polymerization.

Radiopacity of the embolic composition may optionally be achieved by adding a radiopaque agent, such as an aqueous iodinated contrast liquid or an insoluble radiopaque material. It is often desirable that the embolic composition retains its radiopacity after implantation, and thus it is preferable to use relatively insoluble radio pacification agents such as barium sulfate or tantalum powder. For example, if tantalum is used, the tantalum may be provided in a range of about 20 to about 50 percent of a total weight of the embolic composition. As can be appreciated, the radiopaque material may be provided in a variety of other percentages and the present invention is not limited to the preferred range.

The embolic composition may also optionally include a therapeutic agent. The therapeutic agent may be added to the embolic composition in a variety of ways, but is typically bonded to a backbone of the embolic composition or mixed within the embolic composition.

The embolic composition typically has a first viscosity upon delivery into the body lumen and a progressively higher viscosity as the material begins to cure. After the embolic composition has substantially cured, it typically becomes a solid or a gel-like material in vivo. The embolic composition may exhibit, for example, a cure time between about 5 seconds and about 3 minutes, but the cure time may be adjusted to any desired cure time by adding additional components to the embolic composition or by varying the ratios of the components of the embolic composition.

Delivery of the embolic composition or delivery of the individual components may be carried out with a catheter or a syringe. The individual components may be mixed in vitro or in vivo. During delivery of the embolic composition, a flow of bodily fluids through the body lumen may be reduced prior to depositing the embolic composition in the body lumen. For example, reducing a flow of bodily fluids may comprise inflating an occlusion balloon in the body lumen.

The embolic compositions of the present invention typically comprise two or more miscible, chemical components that interact with each other and polymerize in vivo. Some exemplary embolic compositions that may be used with the present invention are in the family of Michael addition polymers.

In one embodiment, the embolic solution comprises polyethylene glycol diacrylate (PEGDA) and pentaerythritol tetra(3-mercaptopropionate) (QT). Some useful embodiments of the PEGDA component of the embolic composition have a molecular weight between about 700 and about 800 and during delivery the embolic composition may have a viscosity between about 5 centipoise and about 3000 centipoise before curing.

In such embodiments, the PEGDA and QT may be provided in a variety of different mass ratios. One preferred mass ratio of PEGDA to QT, when the PEGDGA has a molecular weight of 745, is between about 2 to 1 and about 3 to 1, and preferably about 3 to 1. Such a ratio, while not essential, provides a high degree of cross-linking and provides desirable properties to the embolic composition.

A physiologically acceptable buffer solution, such as glycylglycine, may be mixed with a mixture of the PEGDA and QT for formulations in which it is desirable, by controlling the pH of the buffer, to control the pH of the embolic composition and to modulate the pH effect of the other components of the embolic composition. Optionally, saline may also be added into the embolic composition in order to reduce the viscosity of the embolic composition.

As described above, a radiopaque agent may optionally be added to any of the components prior to mixing the buffer solution with the PEGDA and QT. The radiopaque agent may be insoluble or soluble. Some examples of the radiopaque agent include, but are not limited to, barium sulfate, tantalum, or an iodinated contrast agent. The radiopaque agent may be provided in a range, for example, between about 20 to about 50 weight percent of the embolic composition. The embolic composition may also comprise a therapeutic agent that is contained in the embolic composition as a suspension, a mixture or chemically bonded to one of the components of the embolic composition. The therapeutic agent is typically bonded to a backbone of the embolic composition, and preferably bonded to a PEG backbone or arm of the embolic composition.

In another embodiment of the present invention, the embolic composition comprises a mixture of poly(propylene oxide)diacrylate (also referred to as poly(propylene glycol) diacrylate) (PPODA), and pentaerythritol tetra(3-mercaptopropionate) (QT). A physiologically acceptable buffer solution, such as glycylglycine, may be mixed with the PPODA and QT. Similar to the above example, a radiopaque agent may optionally be added to any of the components prior to mixing the buffer solution with the PPODA and QT. We have found it useful for the radiopaque agent to be insoluble or soluble. Some examples of suitable radiopaque agents are tantalum, barium sulfate and an iodinated contrast agent. The radiopaque agent may be provided in a range between about 20 to about 50 weight percent of the embolic composition. The radiopaque material may be provided in a variety of other percentages and the present invention is not limited to the preferred range. Optionally, a therapeutic agent may be added to the PPODA, QT, and/or buffer solution. The embolic composition may comprise a therapeutic agent that is contained in the embolic composition as a suspension, a mixture or chemically bonded to one of the components of the embolic composition. The therapeutic agent is typically bonded to a backbone or arm of the embolic composition, and preferably bonded to a PEG backbone of the embolic agent. Optionally, saline may be added into the embolic composition to reduce the viscosity of the uncured or liquid embolic composition.

In yet another embodiment, the embolic composition may comprise a mixture of ethoxylated trimethylolpropane triacrylate (ETMPTA) and pentaerythritol tetra(3-mercaptopropionate) (QT). A physiologically acceptable buffer solution, such as glycylglycine, may optionally be mixed with the ETMPTA and QT. A soluble or insoluble radiopaque agent may be added to the any of the components prior to mixing the buffer solution with the ETMPTA and QT. Some examples of suitable radiopaque agents are tantalum, barium sulfate and an iodinated contrast agent. The radiopaque agent may be provided in a range between about 20 to about 50 weight percent of the embolic composition. The embolic composition may comprise a therapeutic agent that is contained in the embolic composition as a suspension, a mixture or chemically bonded to one of the components of the embolic composition. The therapeutic agent typically is bonded to a backbone or arm of the embolic composition, and preferably bonded to a PEG backbone of the embolic agent. Optionally, saline may be added into the embolic composition to reduce a viscosity of the embolic composition.

In a further aspect, the present invention provides a kit for depositing an embolic composition into a body lumen. The kit may comprise an embolic composition, instructions for use, and a delivery device configured to access the body lumen and to deliver the embolic composition to the body lumen.

The embolic composition of the kit may comprise polyethylene glycol diacrylate, pentaerythritol tetra 3(mercaptopropionate) (QT), and a physiologically acceptable buffer solution. Alternatively, the embolic composition of the kit may include ethoxylated trimethylolpropane triacrylate (ETMPTA), QT, and a physiologically acceptable buffer solution, or polypropylene glycol diacrylate or polypropylene oxide diacrylate (PPODA), QT, and a physiologically acceptable buffer solution. Typically, each of the components of the embolic composition is held in separate containers, such as separate syringes.

The delivery device may be a catheter configured to endovascularly access the body lumen or a syringe that is configured to percutaneously access the body lumen.

The kits may further include instructions for use setting forth any of the methods described herein. Optionally, the kits may include an apparatus for combining or mixing the components of the embolic composition prior to delivery into the body lumen. Furthermore, the kits may also include an occlusion assembly for reducing the flow of blood through the body lumen during the embolization procedure. For example, the occlusion assembly may include an occlusion member that is in the form of an inflatable balloon.

The kits may also include packaging suitable for containing the delivery device, embolic composition, and the instructions for use. Exemplary containers include pouches, trays, boxes, tubes, and the like. The instructions for use may be provided on a separate sheet of paper or other medium. Optionally, the instructions may be printed in whole or in part on the packaging. Usually, at least the delivery device will be provided in a sterilized condition. Other kit components, such as a guidewire or an endovascular graft, may also be included.

In yet another aspect, the present invention provides compositions and methods for tissue bulking. Any of the compositions described herein may be used to add bulk to target tissues to aid in functionality or appearance of the target tissue.

These and other aspects of the invention will become more apparent from the following detailed description of the invention when taken in conjunction with the accompanying exemplary drawings.

### BRIEF DESCRIPTION OF THE DRAWINGS

FIGS. 1A to 1D schematically illustrate method of mixing separate components of an embolic composition.

FIGS. 2 to 4 illustrate three exemplary methods of mixing three specific embolic compositions that are encompassed by the present invention.

FIG. 5 illustrates one method of embolizing a body lumen.

FIG. 6 illustrates one method of embolizing an endoleak around an endovascular graft.

FIG. 7 illustrates one method of embolizing an arteriovenous malformation (AVM).

FIG. 8 illustrates a kit of the present invention.

### DETAILED DESCRIPTION OF THE INVENTION

The present invention provides embolic compositions and methods of blocking or obstructing flow through a body lumen. The embolic composition may be delivered to a target site in a body lumen as a low viscosity liquid or, alternatively, as a high viscosity liquid or paste. The embolic composition may move to a progressively higher viscosity as the composition begins to cure or otherwise solidify in vivo to form a solid or gel-like substance.

Numerous clinical applications exist for embolization of both vascular and nonvascular body lumens. The most prevalent uses of the present invention include, but are not limited to, neurological treatment of cerebral aneurysms, AVMs and AVFs, and the peripheral treatment of uterine fibroids and hypervascular tumors. It should be appreciated, however, that the present invention is equally applicable for other uses, such as tissue bulking applications (e.g., aiding functionality of various organs or structures, such as assisting in closing a stricture (including restoring competence to sphincters to treat fecal or urinary incontinence or to treat gastroesophageal reflux disease (GERD)), augmentation of soft tissue in plastic or reconstructive surgery applications (e.g., chin or cheek reshaping), replacing or augmenting herniated or degenerated intervertebral disks, adding structure to or replacing various bursa in and around the knee and elbow, etc.), and in a variety of vascular or non-vascular body lumens or orifices, such as the esophagus, genito-urinary lumens, bronchial lumens, gastrointestinal lumens, hepatic lumens, ducts, aneurysms, varices, septal defects, fistulae, fallopian tubes and the like. Moreover, it should be appreciated that the embolic compositions of the present invention may be used in conjunction with other components, such as endovascular grafts, stents, inflatable implants, fibers, coils, and the like. The embolization materials as taught herein may be used in other applications as identified in co-pending U.S. patent application Ser. No. 10/461,853, entitled “Inflatable Implant” to Stephens et al., the entirety of which is incorporated herein by reference.

There are a variety of advantages of a liquid embolic composition over alternative approaches such as coils or particles. A liquid embolic composition may be delivered to areas of the vasculature inaccessible by coils or particles, and may provide a complete “cast” of a segment of the arterial tree after the embolic composition cures (such as a hypervascular tumor or an AVM), thereby reducing the opportunity for development of collateral perfusion.

The embolic compositions of the present invention have several advantages over other known liquid embolic compositions such as polymer solutions or cyanoacrylates (CAs). First, polymer solutions (such as Onyx® by Micro Therapeutics, Inc., Irvine, Calif.) have precipitation rates that are difficult to control and thereby provide suboptimal filling of an aneurysm sac, AVM, or tumor. Curing of these materials may also be inhibited when solvent concentrations locally increase, such as in an aneurysm sac that is confined by an occlusion balloon. The cure rate of the embolic compositions of the present invention may be easily controlled during the formulation process and the physician may be provided with a range of cure times to meet the needs of various clinical situations. Further, curing of the embolic compositions of the present invention is not adversely affected by a high concentration of embolic composition in a confined region, such as an aneurysm sac, for example. In addition, the present invention provides a dense polymer mass after curing which is less prone to recanalization than the polymer resulting from precipitation approaches. Known CA technologies suffer from difficult delivery procedures with a significant risk of gluing the delivery catheter into the tissue bed and requiring surgical intervention. In addition, some CA technologies have demonstrated poor degradation resistance in vivo and have permitted late recanalization of the embolized lumen.

Finally, no other liquid embolic approach offers the same potential for combining mechanical embolic action with local delivery of a therapeutic agent. The embolic compositions of the present invention include polymers that contain PEG backbones and related molecular structures such as polypropylene glycol and ethoxylated trimethylolpropane, and these materials are good for use as drug delivery agents. There exist known methods to bind a wide range of active therapeutic agents to these materials. Additionally, or alternatively, therapeutic agents may be mixed into the embolic material and subsequently released by difflusion.

The embolic compositions of the present invention may provide desirable mechanical properties that are not provided by the conventional embolic compositions. For example, prior to curing, the liquid embolic compositions may have a high biocompatibility and a controllable solubility which is independent of the environment in which the embolic composition is delivered (e.g., in blood or other bodily fluid). Additionally, the embolic compositions typically have a viscosity of 100 cP or higher, a controllable hydrophobicity, and a low cure time sensitivity to its environment.

After curing, the embolic composition maintains its high biocompatibility and is stable in blood. The cured embolic composition provides desirable mechanical properties such as, a specific gravity between 1.15 to over 1.4, an elastic modulus between about 30 and about 500 psi, a strain to failure of about 25% to about 100% or more, a volume change upon curing between about 0 to about 200% or more, and a water content between less than 5% to greater than about 60%. As can be appreciated the pre-cure properties and post-cure properties of the embolic composition described above are merely examples and should not limit the scope of the embolic compositions of the present invention. The components of the embolic compositions of the present invention may be modified to provide other pre-cure and post-cure mechanical properties, as desired.

One class of suitable embolic compositions that may be used with the present invention is the family of Michael addition polymers formed by combining two or more components under conditions that allow polymerization of the two or more components, where polymerization occurs through a self selective reaction between a strong nucleophile and a conjugated unsaturated bond or conjugated unsaturated group by nucleophilic addition. Such polymers and their reactions are described in International Publication No. WO 00/44808, entitled “Biomaterials formed by Nucleophilic Addition Reaction to Conjugated Unsaturated Groups” to Hubbell, International Publication No. WO 01/92584, entitled “Conjugate Addition Reactions for the Controlled Delivery of Pharmaceutically Active Compounds” to Hubbell et al., U.S. patent application Ser. No. 09/496,231 to Hubbell et al., filed Feb. 1, 2000 and entitled “Biomaterials Formed by Nucleophilic Addition Reaction to Conjugated Unsaturated Groups” and U.S. patent application Ser. No. 09/586,937 to Hubbell et al., filed Jun. 2, 2000 and entitled “Conjugate Addition Reactions for the Controlled Delivery of Pharmaceutically Active Compounds”. The entirety of each of these patent applications and publications are hereby incorporated herein by reference.

As taught in these references, for instance, the components may be a monomer or polymer, such as poly(ethylene glycol), poly(ethylene oxide), poly(vinyl alcohol), poly(ethylene-co-vinyl alcohol), poly(acrylic acid), poly(ethylene-co-acrylic acid), poly(ethyloxazoline), poly(vinyl pyrrolidone), poly(ethylene-co-vinyl pyrrolidone), poly(maleic acid), poly(ethylene-co-maleic acid), poly(acrylamide), or poly(ethylene oxide)-co-poly(propylene oxide) block copolymers. These components may be functionalized to comprise a strong nucleophile or a conjugated unsaturated group or conjugated unsaturated bond. The strong nucleophile may be a thiol or a group containing a thiol, where the conjugated unsaturated group may be an acrylate, an acrylamide, a quinone, or a vinylpyridinium (such as 2- or 4-vinylpyridinium). The functionality of the components may be two, three, or more.

A particular embodiment of a Michael addition polymer useful in the present invention is one formed by the reaction of the functionalized polymer, such as an acrylate polymer, and a multi-thiol nucleophile. These materials can be delivered in liquid or semi-liquid form and may thereafter be crosslinked in vivo to form a “cured” solid or semi-solid gel or gel-like polymer in the target body lumen.

A buffer solution may be optionally be added to the polymer or monomer and nucleophile components. The pH of the buffer solution may be selected to provide the appropriate cure time for the embolic composition. It may also be convenient to adjust the cure time by adjusting any of the strength, amount, and/or pH of buffer solution to provide the user with ample time to deliver the embolic composition to the target site such as a body lumen.

A radiopaque agent may also be added to facilitate visualization of the embolic composition under fluoroscopy and/or follow-up imaging modalities such as computed tomography (CT). Suitable radiopaque agents include relatively insoluble materials such as barium sulfate and tantalum, and soluble materials such as iodinated contrast agents. For example, Applicants have found that it is desirable to use tantalum, typically in the range of about 20 to about 50 weight percent and preferably about 30 weight percent of the total weight of the complete embolic composition, as a radiopaque agent to reduce the late dissipation of radiopacity (due to tantalum's lower solubility in fluids such as water and blood as compared to that of barium sulfate).

Applicants have found that for embolization applications it is desirable to increase the viscosity and hydrophobicity of the uncured material and thereby facilitate controlled placement without unintended embolization of distal vascular beds by reducing or eliminating saline or water from the embolic composition. Reducing the saline and water prior to curing has been found to achieve the best viscosity for delivery into the body lumen, maximizes the degradation resistance of the cured polymer and maximizes the cohesiveness and hydrophobicity of the embolic composition.

Low viscosity formulations of the embolic compositions of the present invention may be used to deeply penetrate tumor vascular beds or other target embolization sites prior to curing of the composition. Occlusion balloons (such as a Swan-Ganz dual-lumen catheter or the EQUINOX™ Occlusion Balloon Catheter manufactured by Micro Therapeutics, Inc. of Irvine, Calif.) or other ancillary flow-blocking devices, such as brushes or other obstructive devices, some of which may be placed on a catheter or stent, such as those sometimes placed across a cerebral aneurysm to be embolized, may be used to prevent flow of the embolic composition beyond the target embolization site.

High viscosity and/or thixotropic (shear-thinning) formulations of these compositions may be used to limit the flow to the neighborhood of the delivery catheter and to facilitate the tendency of the embolic composition to remain in the vicinity of the location in which it was delivered, sometimes even in the presence of substantial blood flow. or other forces. Viscosity and/or thixotropy characteristics may be increased by adding bulking and/or thixotropic agents, such as fumed silica. The bulking agent may be added anytime during the formation of the embolic composition, but is typically preloaded with one of the components, and preferably preloaded with the monomer/polymer or buffer solution.

Some examples of additives that are useful include, but are not limited to, sorbitol or fumed silica that partially or fully hydrates to form a thixotropic bulking agent, and the like. Desirable viscosities for the gels range from about 5 centipoise (cP) for a low-viscosity formulation (such as might be used to deeply penetrate tissue in a hypervascular tumor) up to about 1000 cP or higher for a higher viscosity formulation (such as might be used to treat a sidewall cerebral aneurysm while minimizing the chance of flow disturbance to the embolic composition during the curing process.) As can be appreciated, other embodiments of gels may have a higher or lower viscosity, and the present invention is not limited to such viscosities as described above.

Optionally, the embolic compositions of the present invention may be used to deliver drugs to the target site. The drugs may be mixed in or attached to the embolic composition using a variety of methods. Some exemplary drugs and methods for attaching the drugs to the embolic composition are described in J. M. Harris, “Laboratory Synthesis of Polyethylene Glycol Derivatives,” Journal of Macromolecular Science-Reviews in Macromolecular Chemistry, vol. C-25, No.3, pp. 325-373, Jan. 1, 1985; J. M. Harris, Ed., “Biomedical and Biotechnical Applications of Poly(Ethylene Glycol) Chemistry”, Plenum, N.Y., pp. 1-14, 1992; Greenwald et al., “Highly Water Soluble Taxol Derivatives: 7-Polyethylene Glycol Carbamates and Carbonates:”, J. Org. Chem., vol. 60, No. 2, pp. 331-336, 1995, Matsushima et al., “Modification of E. Coli Asparaginase with 2,4-Bis(O-Methoxypolyethylene Glycol)-6-Chloro-S-Triazine (Activated PEG.sub.2); Disapperance of Binding Ability Towards Anti-Serum and Retention of Enzymic Activity,” Chemistry Letters, pp. 773-776, 1980; and Nathan et al., “Copolymers of Lysine and Polyethylene Glycol: A New Family of Functionalized Drug Carriers,” Bioconjugate Chem. 4, 54-62 (1993), each of which are incorporated herein by reference.

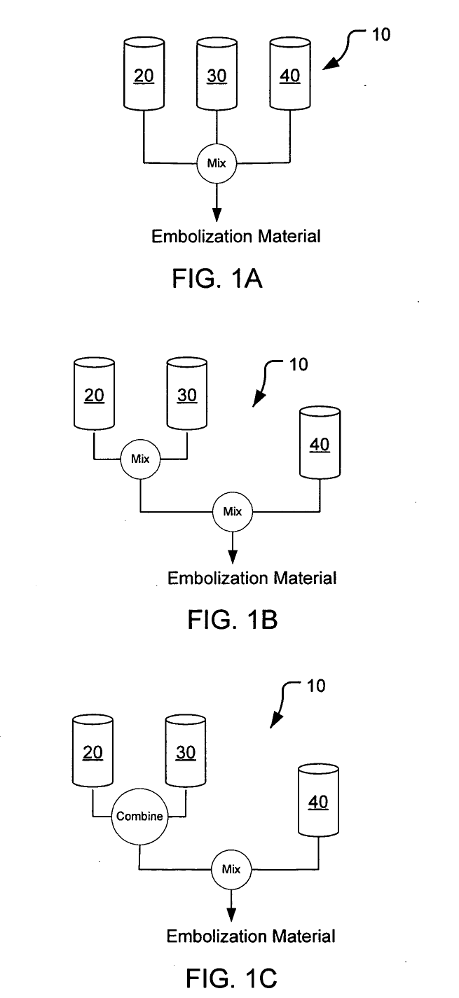

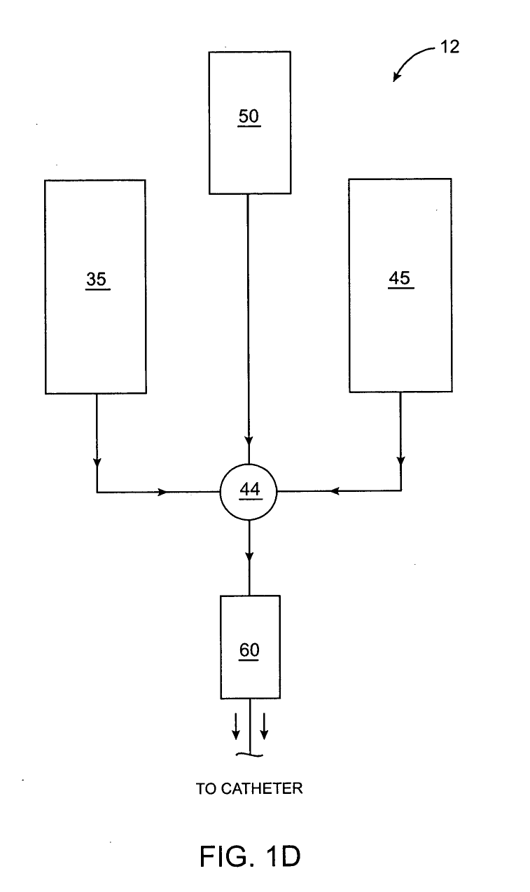

The three (or more) components of the embolic compositions of the present invention may be mixed any number of ways, including by way of example, only by hand, with two or more syringes, or with a mixing apparatus (not shown). FIGS. 1A to 1C illustrate some methods that may be used to form the embolic compositions of the present invention. As can be appreciated, FIGS. 1A to 1C are merely examples; the present invention is not limited to such methods.

Referring now to FIG. 1A, the three chemical components (monomer or polymer, nucleophile, and buffer) may be packaged separately in sterile syringes 20, 30, 40. Each of the syringes 20, 30, 40 may be coupled to a mixing apparatus and each of the components may be thoroughly mixed together. The resulting three-component liquid embolic composition is then ready for introduction into the target site in the body lumen, as it will cure into a gel having the desired properties within the next several minutes or other desired cure time.

In another method shown in FIG. 1B, two of the components (e.g., QT and buffer—typically glycylglycine) are first thoroughly mixed, typically between their respective syringes 20, 30 for a sufficient time (e.g., about two minutes). The third component (e.g., a Michael addition polymer, such as PEGDA) is then thoroughly mixed in from syringe 40 with the resulting two-component mixture for a time sufficient to ensure adequate mixing and to form the embolic composition (e.g., approximately three minutes). This resulting three-component mixture is then ready for introduction into the target site in the body lumen as it will cure into a gel having the desired properties within the next several minutes or other desired cure time.

In the method shown in FIG. 1C, two of the components (e.g., QT and the buffer) are combined (not mixed as with the example of FIG. 1B). In the FIG.1C example, the term “combined” indicates the act of transferring the contents of syringe 20 to syringe 30 (or vice versa), with relatively little agitation (e.g., “ping-ponging”) such that the resulting combination may not necessarily be a homogeneous or near-homogeneous mixture. After the two components are combined, the combined components are thoroughly mixed with the third component (e.g., monomer or polymer) for a time sufficient to ensure adequate mixing and to form the embolic composition (e.g., approximately three minutes). This resulting three-component mixture is then ready for introduction into the target site in the body lumen as it will cure into a gel having the desired properties within the next several minutes or other desired cure time. Cure times of the embolic composition may be tailored by adjusting the formulations, mixing protocol, and other variables according to the requirements of the clinical setting. Details of suitable delivery protocols for these materials in the particular application of filling an inflatable endovascular graft are discussed in copending U.S. Pat. No. 6,761,733 to Chobotov et al. entitled “Delivery Systems and Methods for Bifurcated Endovascular Graft” and Published U.S. patent application Ser. No. 10/327,711 to Chobotov et al., the complete disclosures of which are incorporated herein by reference. Applicants have found the post-cure mechanical properties of these gels to be highly tailorable without significant changes to the formulation. For instance, these gels may exhibit moduli of elasticity ranging from tens of psi to several hundred psi; the formulation described above exhibits moduli ranging from about 175 to about 250 psi with an elongation to failure ranging from about 30 to about 50 percent.

One specific example material suitable for this embolization application is a Michael addition polymer formed by mixing polyethylene glycol diacrylate (PEGDA) with pentaerythritol tetra(3-mercaptopropionate) (QT). A physiologically acceptable buffer solution, such as glycylglycine, N-[2-hydroxyethyl]piperazine-N′-[2-ethanesulfonic acid] (HEPES), or other suitable buffer solution may optionally be added to adjust the solidification time and/or the viscosity of the liquid components prior to curing.

As a specific example, a low viscosity formulation of embolic composition using PEGDA with a molecular weight (MW) of about 745 and a mass ratio of PEGDA to QT of between about 2 to 1 and about 3 to 1 is particularly appropriate, along with about 10 weight percent to about 25 weight percent of 400 millimolar glycylglycine buffer and a cure time of between about 1 minute and about 3 minutes, preferably between about 1 minute and about 2 minutes. As shown in FIGS. 1A to 1C, this formulation may be described as a system 10 in which each of the three components PEGDA, QT and glycylglycine are packaged in separate containers, such as syringes 20, 30, 40, respectively. About 30 weight percent tantalum powder, may optionally be added to any of the components. In one embodiment, the tantalum powder has an average particle size of less than about 5 microns. In other embodiments, other radiopaque markers or the tantalum powder having a larger or smaller average particle size may be used. Tantalum powder meeting these requirements can be procured from numerous commercial sources, such as Sigma-Aldrich Inc., St. Louis, Mo.

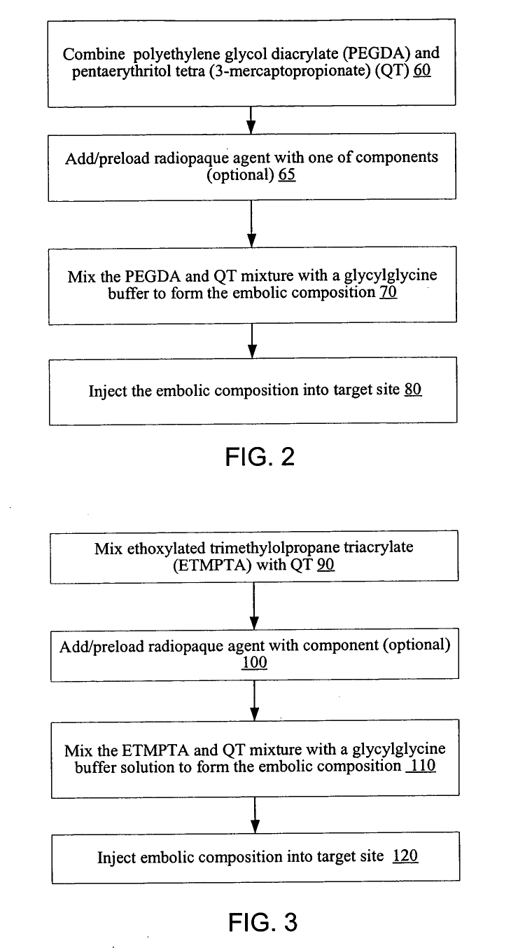

FIG. 2 illustrates one exemplary method of preparing the embolic composition described above in conjunction with FIG. 1C for delivery into the body lumen. The PEGDA and QT are first combined by transferring back and forth all of the material as discussed in connection with FIG. 1C into one syringe, step 60. Optionally, the radiopaque agent, such as tantalum or barium sulfate, may be preloaded with one of the components, or otherwise added to the mixture of the PEDGA and QT, step 65.

Thereafter, the PEGDA and QT combination may be mixed with the glycylglycine buffer (and radiopaque agent) by connecting the two syringes, step 70. In one embodiment the two syringes are connected through a 3-way adapter and the components are mixed by “ping-ponging” the material from one syringe to the other for about 15 to about 30 seconds. Different formulations of the component materials may require different mixing times. After the components have been mixed for a sufficient time, the embolic composition may be injected into a target site in the body lumen immediately after the completion of mixture of the three components, step 80. It may be convenient to transfer the material in 1 cc increments to a 1 cc syringe to reduce the operator effort when injecting the material through a microcatheter with a lumen less than about 0.025″. As noted above, it may also be convenient to adjust the cure time by adjusting any of the strength, amount, and/or pH of the buffer solution to provide the user with ample time to deliver the embolic composition to the target site such as a body lumen 80. As can be appreciated, the above example is merely illustrative and a variety of other conventional and proprietary methods of mixing the embolic composition may be used. Moreover, it should be appreciated that any of the embolic compositions described herein may be mixed using the above described method of forming the embolic composition.

FIG. 3 illustrates an example of another class of chemical embolic compositions of the present invention. In this example, a polymer is formed by mixing ethoxylated trimethylolpropane triacrylate (ETMPTA) with the QT, step 90, as described above. Specifically, for QT with a molecular weight of 488.7 and ETMPTA with a molecular weight of 956, a QT/ETMPTA mass ratio between about 0.38 and 0.50 is useful. Glycylglycine should represent between about 10 weight percent and about 50 weight percent of the mixture, with pH adjusted to achieve the desired cure time.

Similar to above, radiopacity of embolic composition may be achieved by optionally adding an aqueous iodinated contrast liquid or an insoluble radiopaque material, such as barium sulfate or tantalum powder, as described above for the PEGDA-QT embolic composition. The radiopaque agent may be preloaded with any of the components, or otherwise mixed with the three components, step 100.

The buffer solution, if used, may be mixed with the ETMPTA and QT mixture to form the embolic composition, step 110. One suitable buffer solution for this embolic composition is glycylglycine, adjusted to a pH to yield the desired cure time. Higher buffer pH results in a faster crosslinking reaction and therefore shorter cure time. Once the components have been mixed, the embolic composition may be delivered to the target site, such as a body lumen, step 120.

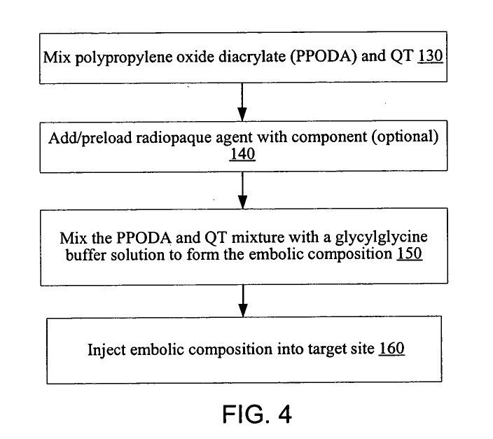

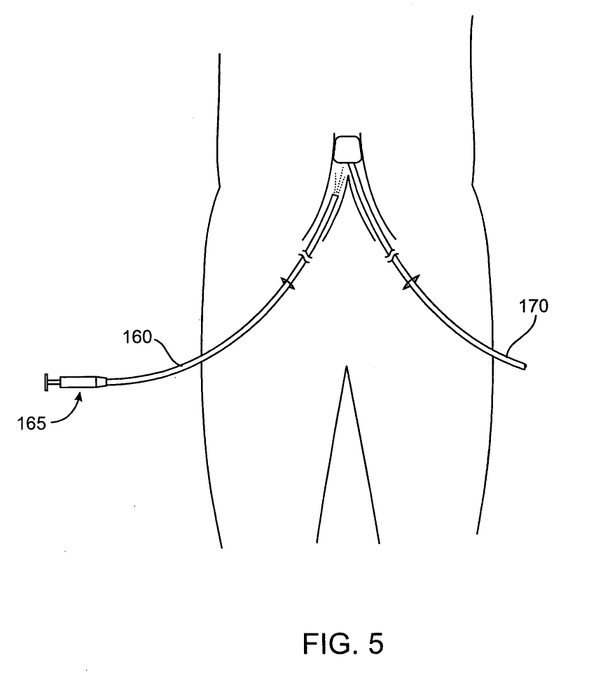

FIG. 4 illustrates an example of yet another class of chemical embolization components of the present invention. At step 130, a polymer precursor is formed by mixing PPODA (alternatively, polypropylene glycol diacrylate) with QT. To add radiopacity to the resultant embolic composition, a radiopaque agent optionallymnay be preloaded with any of the components, or otherwise added to the embolic composition, step 140. A buffer solution (e.g., glycylglycine ) may then be mixed with the PPODA and QT mixture to form the embolic composition, as described above, step 150. Thereafter, the embolic composition may be injected into the target site such as body lumen, step 160.

A potential advantage of this material is that PPODA is much more hydrophobic than either PEGDA or ETMPTA, may have less tendency to disperse into the blood at a given viscosity and therefore may be less likely to produce unintended distal embolization. Another potential advantage is that the embolic material utilizing PPODA generally has a higher elastic modulus than either PEGDA or ETMPTA, which may be useful in applications such as tissue bulking, for instance, in which a stiffer material is desirable. A particularly useful formulation comprises PPODA (e.g., Aldrich 45,502,4 manufactured by Sigma-Aldrich, Inc. of St. Louis, Mo.) having a molecular weight of approximately 900 and QT having a molecular weight of 488.7; the QT/PPODA mass ratio ranging from about 0.25 to 0.40 and glycylglycine added to comprise between about 5 weight percent and 40 weight percent of the entire mixture. Other buffers may be used to adjust the pH to achieve the desired cure time.

The embolic compositions of the present invention may be delivered via an endoluminal catheter to the desired site of embolization. Alternatively, the embolic composition may be delivered via a needle or other external puncture device. Some examples of suitable catheters include those with a lumen generally greater than about 0.014″, such as, e.g., the REGATTA®, FASTRACKER®, PROWLER®, TURBOTRACKER®, TRACKER®, EXCEL™, RAPID TRANSIT®, RENEGADE™, REBAR™, MASS TRANSIT®, HI-FLO™, GT LEGGIERO™, and EMBOCATH™ products.

Desirable characteristics of a catheter for delivering the embolic compositions of the present invention include those that facilitate positioning the catheter tip at the desired point in the target site for embolization (e.g., atraumatic flexible tip, pushable, torqueable and trackable shaft, adequate radiopacity, and the like). The embolic compositions disclosed here are generally compatible with a wide range of catheters in clinical use and do not require the use of specialized catheter materials (as do certain alternative embolic technologies such as those using dimethyl sulfoxide (DMSO)). To minimize the effort required to inject the embolic composition into the body lumen, the catheter length should be chosen to be as short as feasible for reaching the embolization target site.

Materials such as those described above are typically mixed immediately prior to use. This mixing can be easily accomplished in less than a minute by transferring the material back and forth between two syringes connected by, for example, a 3-way stopcock. If larger quantities, for example greater than 5 ml, are desired, a mixing device such as described in commonly owned, copending U.S. patent application Ser. No. 10/658,074, entitled “Fluid Mixing Apparatus and Method,” filed Sep. 8, 2003, the complete disclosure of which is incorporated herein by reference, may be used to accomplish the mixing. It should be appreciated, however, that if desired, the components of the embolic composition may be chosen such that the cure time of the embolic composition is longer. This allows the user to premix the embolic composition components, thus allowing more time to deliver the embolic composition into the target site.

In many situations where larger quantities of embolic composition are needed, it may be useful progressively to mix and inject materials of the present invention from each of several kits and perform angiography after each injection to assess the incremental progress of the treatment and to highlight where any additional embolic composition might be placed, if any.

Using the approaches described above, in which either the viscosity of the embolic composition or adjunctive devices such as occlusion balloons are used to prevent unintended distal flow of the embolic composition, the cure time may be tailored to provide sufficient time for the clinician to deliver the material to the target embolization site after mixing but before curing progresses to the point that delivery becomes difficult due to the concomitant increasing viscosity of the mixture. The advantages of this approach are the simplicity of the delivery system and the ease with which larger volumes of embolic composition can be delivered.

Useful quantities of embolic compositions of the present invention range from a low of about 0.5 ml to about 1.0 ml for small neurovascular aneurysm applications up to about 30 ml for treating stent graft endoleaks. Even more, up to 100 ml or more, may be used for example in treating stent graft endoleaks in cases where the entire aneurysm sac may be filled with embolic composition, such as may be the case with AAAs. When the embolic composition is radiopaque, material may be deposited in stages with angiography used to evaluate the need and target location for any additional quantity of the embolic composition to achieve the therapeutic objective.

Alternatively, instead of mixing the components in vitro as described above, components of the embolic compositions, may be mixed at the time of use by delivering the components through separate catheter lumens to a mixing device (e.g., a static mixer) located at a distal end of a delivery system. Some examples of static mixers are manufactured by ConProTec Inc. of Salem, N.H. under the name STATOMIX®. The components of the polymer embolic composition may be mixed in this device by pushing the separate components of the embolic composition through the catheter just before delivery to the target site for embolization

In such a case, a static mixer may be located for example at the proximal end of the delivery catheter such as shown schematically in FIG. 1D. This exemplary configuration has a number of clinical advantages when embolizing, e.g., an AVM, in which it is helpful to incrementally inject small volumes of embolization material into the site followed by injecting contrast therein so that the clinician may determine the pathway and extent to which the embolization material has entered, in this example, the AVM's vascular network. Repeating this pattern of alternatively injecting embolization material and contrast until the clinician is satisfied that only the necessary amount of embolization material has been used may result in a safer and more efficacious clinical outcome.

In the schematic exemplary configuration of FIG. 1D, system 12 is shown as comprising a source of embolic composition components, in this case containers or syringes 35 and 45. In this example, the contents of syringe 35 contains two of the components (e.g., QT and buffer—typically glycylglycine) while syringe 45 contains the third component (e.g., a Michael addition polymer, such as PEGDA). Syringes 35 and 45 are connected to a four-way valve 44 which is also connected to a source 50 of radiopaque contrast material such as that used for performing an angiography. The output of valve 44 leads to a static mixer 60 which is in turn connected to the delivery catheter (not shown). Three or more embolic composition component containers or syringes connected to a multi-path valve as described herein may also be used.

In an example of how the FIG. 1D embodiment of a proximal end static mixer apparatus may be used to treat, e.g., an AVM, the clinician will use conventional techniques to gain delivery catheter access to the AVM site into which the embolic composition is to be introduced. Valve 44 is set so that the contents of only syringes 35 and 45 may be transferred through valve 44 to mixer 60 while preventing the introduction of any contrast material from source 50 into mixer 60. The contents of syringes 35 and 45 may be transferred through static mixer 60 into the delivery catheter and subsequently to the target site in the body.

Next, valve 44 may be adjusted to allow only contrast material from source 50 through mixer 60 (while preventing the introduction of any material from syringes 35 or 45) into the AVM via the delivery catheter. In this mode the static mixer 60 merely acts as a conduit as no mixing operation is necessary. This feature allows the clinician to interrogate the AVM site and determine, among other things, if a clinically adequate volume of embolic composition has been introduced into the AVM, the composition's path through the AVM vasculature, and how much (if any) additional embolic composition should be injected into the AVM. Using contrast in this manner has the added benefit of ensuring that any embolic composition remaining in system 12 distal to valve 44 is clear before the composition has a chance to cure and otherwise block the mixer 60 and delivery catheter from being able to introduce additional embolic material as described below should the clinician determine it necessary.

If the clinician determines that additional embolic material should be introduced into the AVM, valve 44 may be switched back to its original position so that additional material from containers 35 and 45 (or new containers) may be introduced into the AVM as described above, followed again by adjusting the position of valve 44 as described above to enable only the injection of contrast through valve 44, mixer 60, the delivery catheter, into the AVM. This process of alternatively injecting embolic material in known volumes into the target site followed by the injection of contrast therein may be repeated as many times as necessary to achieve the desired clinical outcome.

It should be understood that the configuration of FIG. 1D is but one of a number of ways this processing technique to embolize target sites of body lumens as described herein may be achieved; thus, the present invention is not limited to this particular configuration.

The in vivo mixing is generally considered to be adequate if a gel is formed with a consistent cure time. Inadequate mixing is typically indicated by failure of the mixture to solidify into a gel, usually due to separation of the hydrophilic and hydrophobic components prior to formation of sufficient crosslinks to hold the components together, wide variation in the cure time for a given formulation, and/or increased gel degradation rate due to nonhomogenities in crosslinking and/or suboptimal polymer morphology. The in vivo mixing does not require pre-mixing by the clinician and may allow the use of a very short cure time (such as between about 5 seconds to about 60 seconds) which may prevent the material from flowing distally beyond the end of the delivery system. The in vivo mixing could also yield a material that has curing behavior similar to that of n-butyl cyanoacrylate materials in current widespread use for embolization.

As noted above, the embolic compositions such as those described above may also be enhanced with therapeutic agents to improve their effectiveness in treating certain disease states. In such embodiments, the embolic composition serves a dual role of acting as a mechanical obstruction to reduce or block the flow of a fluid through a lumen, and acting as a reservoir of therapeutic agent for local delivery to the region of the target embolization site. In this case the embolic composition is placed and allowed to cure, as described above. The therapeutic agent is then released and may be selected to promote thrombosis to reduce the risk of leaks around the embolic composition and/or to provide other therapeutic benefits to the tissue surrounding the device.

In this dual-role embodiment, the therapeutic agent may initially be contained throughout the volume of the embolic composition, and may be contained either as a suspension, a mixture, or by being chemically bonded to one of the components of the embolic composition. The therapeutic agent may be bonded to the backbone or arm of a component of the embolic composition. For example, the therapeutic agent can be bonded to the PEG backbone. Methods for binding therapeutic agents to PEG for delivery at a targeted rate are known. Therapeutic agent could be mixed in with one of the components during manufacturing or could be stored separately and mixed with the other polymer components prior to use.

One particularly beneficial use of the dual-role embodiment is in treating tumors. In such a case, a chemotherapeutic agent is bound to or mixed with the liquid polymer prior to use. The embolic composition is then delivered via catheter into the major arteries feeding the tumor. The embolic composition then flows throughout the vasculature of the tumor and essentially forms a “cast” as it solidifies, thereby making the tumor highly unlikely to recanalize as can happen when particulate embolic technologies are used. Once in place, the polymer begins to release the chemotherapeutic agent into the tumor tissue, enhancing tissue necrosis and/or shrinkage. The embolic composition properties, such as viscosity and thixotropy, are selected to prevent the liquid polymer from passing through the capillary bed of the tumor and exiting into the venous circulation.

One example application of the embolic composition with a therapeutic agent is the treatment of hypervascular tumors. The embolic composition serves to kill the tumor by blocking its supply of blood while also locally delivering a chemotherapeutic agent that further targets and kills cells of the malignancy. Candidate drugs are those with efficacy when delivered intratumorally and may include, for example, traditional agents such as cyclophosphamide, fluorouracil and methotrexate, as well as newer anticancer agents such as doxorubicin, cisplatin and others.

### EXAMPLES

The embolic compositions of the present invention typically are used by placing it in the body at the desired embolization target location. The material then blocks or reduces fluid flow in the body lumen. Several specific examples are described below.

The present invention may be used to embolize, or block blood flow in an artery. As shown in FIG. 5, this may be accomplished by introducing a delivery catheter into the arterial tree at a location remote from the desired embolization site and advancing the catheter to the target site over a guidewire. For example, the delivery catheter 160 can be inserted into the common femoral artery and advanced up to position the tip for embolizing the internal iliac artery. The embolic composition can then be mixed using any of the mixing methods described above. A syringe 165 containing the liquid embolic material can then be attached to the delivery catheter and the liquid embolic material injected directly into the internal iliac artery under fluoroscopic guidance. If focal embolization of the internal iliac is desired (as would typically be the case), an occlusion balloon catheter 170 can be placed in the common iliac artery from a contralateral femoral access and inflated to temporarily stop blood flow into the embolization site while the liquid embolic composition cures. Alternatively, a sufficiently viscous or thixotropic form of the embolic composition can be used such that flow occlusion is not necessary.

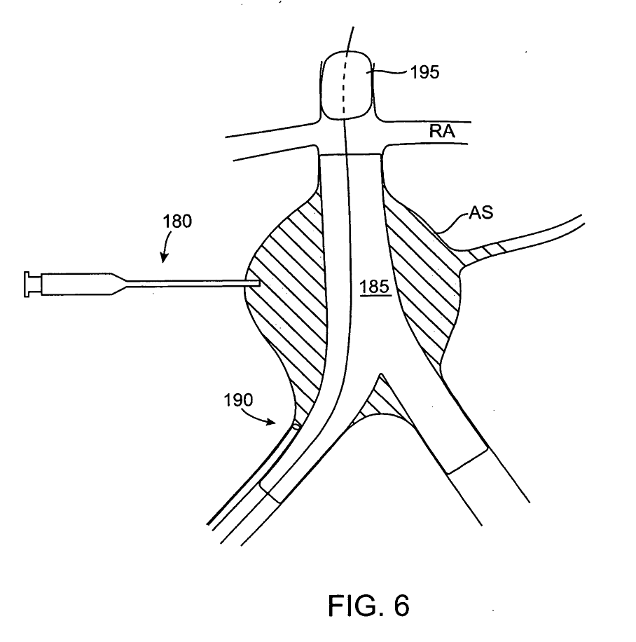

In another use of the invention, as shown in FIG. 6, a translumbar needle 180, sheath or microcatheter 190 can be placed directly into a sac AS of an abdominal aortic aneurysm and the aneurysm sac filled with embolic composition to prevent or eliminate retrograde perfusion of the sac (e.g. a “Type II endoleak”) when an aortic stent graft 185 is placed across the aneurysm. If desired, an occlusion member 195 may be positioned in the aorta to block the blood flow through the aneurysm sac during the embolization procedure. There are numerous commercially available kits suitable for translumbar embolization; one example is a 6 Fr translumbar arteriography puncture kit from Cook Inc. of Bloomington, Ind. A more complete description of delivering an embolic composition into an aneurysm sac may be found in copending and commonly owned U.S. patent application Ser. No. 10/691,849, filed Oct. 22, 2003, the complete disclosure of which is incorporated herein by reference.

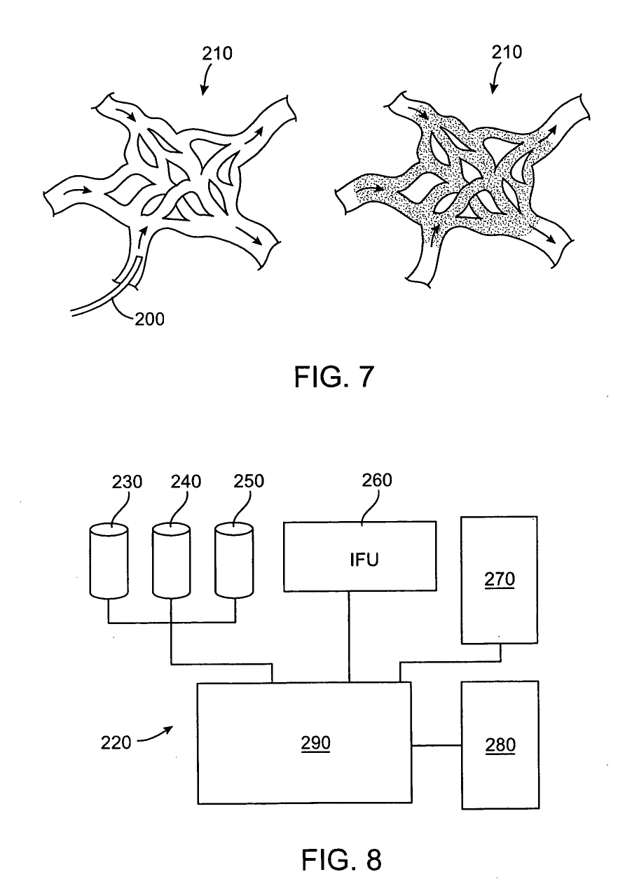

In another example, the present invention can be used to embolize AVMs in the peripheral or neurological vascular beds. As shown in FIG. 7, a delivery catheter 200 is placed at the arterial entrance to the AVM 210 and embolic composition is slowly injected and allowed to solidify to block flow through the AVM. Again, it may be desirable to occlude flow through the AVM until the material has cured to prevent unintended distal flow of the material. Alternatively, a more viscous formulation may be used that remains in the AVM without the necessity of occluding the inflow.

For the embolization of AVMs, two approaches can be used and slightly different optima exist for the associated embolic composition. In one approach, blood flow through the AVM is substantially reduced or halted during the embolization procedure, typically through the use of a proximal occlusion balloon. A low viscosity embolic composition formulation is ideal for this approach in that it can flow easily into most or all of the pedicles of the AVM and provide a complete embolization that is resistant to recanalization. It is particularly difficult to achieve this degree of embolization using particle embolization technologies. In the second approach, blood flow through the AVM is not significantly restricted during the procedure, and a higher viscosity embolic composition formulation is preferable to reduce the potential that some embolic composition flows through the AVM and provides an unintended distal embolus. For this approach, viscosities in the range of about 500 cP to about 3000 cP are preferable.

In yet another example, the present invention may be used to treat cerebral aneurysms. The aneurysm sac is filled with the embolic composition, delivered via a small diameter catheter under fluoroscopic guidance, to exclude it from hemodynamic pressure and thereby eliminate the risk of rupture. The desirable characteristics are the same as above for AVM embolization, except that for this application it is also particularly desirable that the mixed and uncured embolic composition be hydrophobic so that it remains cohesive in the aneurysm sac and does not disperse prior to curing.

The present invention may also be used for embolization of nonvascular body lumens and in tissue bulking applications (as described above) in much the same way as described above for vascular embolization. For example, a delivery conduit (which could be a catheter or a needle or a sheath used with a translumbar needle) is placed with its distal end at the site of the target embolization, the embolic composition is prepared by premixing (if needed), and the embolic composition may then delivered to the target site under fluoroscopic guidance.

In another aspect, the present invention provides kits for delivering the embolic composition to the body lumen. The kits may include any of the embolic compositions described above. Typically, the embolic compositions may be stored in separate syringes/vials. For example, as illustrated in FIG. 8, kit 220 may include the separate components of the embolic composition may be stored in separate syringes 230, 240, 250. Kit 220 may also include instructions for use 260 which sets forth any of the methods described above. One or more delivery devices 270 (described above) may be included in the kit to facilitate delivery of the embolic composition into the desired body lumen. The delivery device may include a built-in mixing apparatus. Alternatively, the kit 220 may include a separate mixing apparatus 280 (described above).

Kit 220 may include a package 290 to hold the components of kit 220. Package 290 may be any conventional medical device packaging, including pouches, trays, boxes, tubes, or the like. The instructions for use 260 will usually be printed on a separate piece of paper, but may also be printed in whole or in part on a portion of the package 290. Optionally, kit 220 may include a guidewire (not shown) for assisting in the positioning of a catheter delivery device for the embolic composition, an endovascular graft, and/or a delivery system for delivering the endovascular graft (not shown).

While particular forms of the invention have been illustrated and described, it will be apparent that various modifications can be made without departing from the spirit and scope of the invention.

1. A method of embolizing a body lumen comprising:
depositing a liquid embolic composition into the body lumen; and
allowing the embolic composition to cure in the body lumen so as to embolize the body lumen,
wherein the embolic composition cures by cross-linking or polymerization.

2. The method of claim 1 wherein the cross-linking or polymerization occurs via a Michael addition process.

3. The method of claim 1 wherein embolic composition cures through a self selective reaction between a strong nucleophile and a conjugated unsaturated bond or conjugated unsaturated group.

4. The method of claim 2 wherein the cross-linking or polymerization occurs by combining a functionalized polymer with a nucleophile.

5. The method of claim 4 wherein the functionalized polymer is an acrylate polymer.

6. The method of claim 5 wherein the nucleophilic compound is a multi-thiol nucleophile.

7. The method of claim 1 wherein the embolic composition comprises a therapeutic agent.

8. The method of claim 7 wherein the therapeutic agent is bonded to a backbone of the embolic composition.

9. The method of claim 7 wherein the therapeutic agent is mixed with or suspended in the embolic composition.

10. The method of claim 1 wherein the embolic composition further comprises a radiopaque agent.

11. An embolic composition comprising:
polypropylene glycol diacrylate (PPODA);
pentaerythritol tetra(3-mercaptopropionate); and
a physiologically acceptable buffer solution.

12. The embolic composition of claim 11 further comprising a radiopaque agent.

13. The embolic composition of claim 11 wherein the embolic composition further comprises a therapeutic agent.

14. An embolic composition comprising:
ethoxylated trimethylolpropane triacrylate (ETMPTA);
pentaerythritol tetra(3-mercaptopropionate); and
a physiologically acceptable buffer solution.

15. The embolic composition of claim 14 further comprising a radiopaque agent.

16. The embolic composition of claim 14 wherein the embolic composition further comprises a therapeutic agent.

17. An embolic composition comprising:
polyethylene glycol diacrylate (PEGDA);
pentaerythritol tetra(3-mercaptopropionate); and
a physiologically acceptable buffer solution.

18. The embolic composition of claim 17 further comprising a radiopaque agent.

19. The embolic composition of claim 17 wherein the embolic composition further comprises a therapeutic agent.

20. An embolic composition formed by in vivo polymerization by a Michael addition process.
